(19) United States
(12) Patent Application Publication
(10) Pub. No.: US 2026/0228561 A1
(43) Pub. Date: Aug. 6, 2026

(54) Deterministic Inference for Machine Learning Models with Variable Behavior

(71) Applicant: Google LLC, Mountain View, CA (US)

(72) Inventors: Atilla Peter Kiraly, San Jose, CA (US); Elzbieta Gruzewska, London (GB); Arnav Agharwal, San Francisco, CA (US); Zaid Isam Nabulsi, San Francisco, CA (US); Scott Mayer McKinney, Oakland, CA (US)

(21) Appl. No.: 19/152,843

(22) PCT Filed: Feb. 3, 2023
(86) PCT No.: PCT/US2023/012323

§ 371 Date: Jul. 31, 2025

(51) Int. Cl. G06N 3/0985

(52) U.S. Cl. CPC G06N 3/0985

(57) ABSTRACT

Provided are systems and methods that enable deterministic inference for machine learning models with variable behavior. In particular, the present disclosure relates to a system in which a machine-learned model has a variable processing portion that is configured to variably apply one or more of a plurality of different processing operations when processing an input. According to an aspect of the present disclosure, one or more seed values can be used to deterministically control which of the plurality of different processing operations are applied by the machine-learned model when processing a given set of input data.

### FIELD

The present disclosure relates generally to machine learning. More particularly, the present disclosure relates to deterministic inference for machine learning models with variable behavior.

### BACKGROUND

It has recently been shown that machine-learned models which are configured to provide variable processing behavior can provide improved performance (e.g., increased model accuracy on a set of test data) as compared to models which have only a single processing configuration. Specifically, multiple instances of a machine-learned model can be used to process the same input data, but with different processing behavior performed by each instance of the model, thereby generating multiple different intermediate outputs (or “inferences”) which can be aggregated, e.g. ensembled, to form an aggregated output. In many settings, the aggregated output is superior in performance to most or all of the different individual outputs. Instead of utilizing multiple independent models for ensembling, randomness in models can be used as a form of multiplexing for ensembling, where a single model is run multiple times and the predictions are aggregated (e.g., averaged) for the purposes of improved performance. Combinations of multiple models with randomness are also possible for improved performance.

However, although the approaches described above which leverage variable processing behavior of a machine learning model at inference time can provide certain benefits (e.g., increased accuracy), they also introduce a number of drawbacks. As one example, machine learning models with variable processing behavior exhibit reduced reproducibility and consistency. In particular, the aggregated prediction produced from a model with variable processing behavior can vary if the underlying variable processes are randomly applied and/or unconstrained. Thus, the same model may produce different results when given the same input (e.g., especially when a continuous-valued output is categorized). This unpredictable behavior is undesirable in a number of settings (e.g., medical devices or other critical applications) in which reproducibility or consistency are of high importance.

### SUMMARY

Aspects and advantages of embodiments of the present disclosure will be set forth in part in the following description, or can be learned from the description, or can be learned through practice of the embodiments.

One example aspect of the present disclosure is directed to a computer system that performs deterministic inference for a machine-learned model with variable behavior. The computer system includes one or more processors and one or more non-transitory computer-readable media that collectively store: the machine-learned model, wherein the machine-learned model is configured to variably apply one or more of a plurality of different processing operations when processing an input; and instructions that, when executed by the one or more processors, cause the computer system to perform inference-time operations. The inference-time operations include: obtaining input data and one or more seed values associated with the input data; processing the input data and the one or more seed values with the machine-learned model to generate one or more inferences from the input data, wherein the one or more seed values deterministically control which of the plurality of different processing operations are applied by the machine-learned model when processing the input data; and providing the one or more inferences as an output.

Another example aspect of the present disclosure is directed to a computer-implemented method for deterministic inference, the method performed by a computing system comprising one or more computing devices. The method includes obtaining input data and one or more seed values associated with the input data. The method includes accessing a machine-learned model, wherein the machine-learned model is configured to variably apply one or more of a plurality of different processing operations when processing an input. The method includes processing the input data and the one or more seed values with the machine-learned model to generate one or more inferences from the input data, wherein the one or more seed values deterministically control which of the plurality of different processing operations are applied by the machine-learned model when processing the input data. The method includes providing the one or more inferences as an output.

Another example aspect of the present disclosure is directed to one or more or more non-transitory computer-readable media that collectively store: a machine-learned model, wherein the machine-learned model is configured to variably apply one or more of a plurality of different processing operations when processing an input; and wherein machine-learned model is configured to receive one or more seed values as input, wherein the one or more seed values deterministically control which of the plurality of different processing operations are applied by the machine-learned model when processing the input.

Another example aspect of the present disclosure is directed to a computer system that performs deterministic inference for a predictive model with variable behavior. The computer system includes one or more processors and one or more non-transitory computer-readable media that collectively store: the predictive model, wherein the predictive model is configured to variably apply one or more of a plurality of different processing operations when processing an input; and instructions that, when executed by the one or more processors, cause the computer system to perform inference-time operations. The inference-time operations include: obtaining input data and one or more seed values associated with the input data; processing the input data and the one or more seed values with the predictive model to generate one or more inferences from the input data, wherein the one or more seed values deterministically control which of the plurality of different processing operations are applied by the predictive model when processing the input data; and providing the one or more inferences as an output. The predictive model can be a machine-learned model or can be a manually-parameterized model.

Other aspects of the present disclosure are directed to various systems, apparatuses, non-transitory computer-readable media, user interfaces, and electronic devices.

These and other features, aspects, and advantages of various embodiments of the present disclosure will become better understood with reference to the following description and appended claims. The accompanying drawings, which are incorporated in and constitute a part of this specification, illustrate example embodiments of the present disclosure and, together with the description, serve to explain the related principles.

### BRIEF DESCRIPTION OF THE DRAWINGS

Detailed discussion of embodiments directed to one of ordinary skill in the art is set forth in the specification, which makes reference to the appended figures, in which:

FIG. 1 depicts a block diagram of example techniques to perform deterministic inference for a machine-learned model with variable behavior according to example embodiments of the present disclosure.

FIG. 2 depicts a block diagram of example techniques to perform deterministic inference for a machine-learned model with variable behavior according to example embodiments of the present disclosure.

FIG. 3 depicts a block diagram of example techniques to perform deterministic inference and aggregate for a set of machine-learned models with variable behavior according to example embodiments of the present disclosure.

FIG. 4 depicts a block diagram of an example technique to determine a set of optimal seed values according to example embodiments of the present disclosure.

FIG. 5 depicts a block diagram of an example technique to implement deterministic inference for a machine-learned model with variable behavior according to example embodiments of the present disclosure.

FIG. 6A depicts a block diagram of an example computing system according to example embodiments of the present disclosure.

FIG. 6B depicts a block diagram of an example computing device according to example embodiments of the present disclosure.

FIG. 6C depicts a block diagram of an example computing device according to example embodiments of the present disclosure.

Reference numerals that are repeated across plural figures are intended to identify the same features in various implementations.

### DETAILED DESCRIPTION

Generally, the present disclosure is directed to systems and methods that enable deterministic inference for machine learning models with variable behavior. In particular, the present disclosure relates to a system in which a machine-learned model has a variable processing portion that is configured to variably apply one or more of a plurality of different processing operations when processing an input. According to an aspect of the present disclosure, one or more seed values can be used to deterministically control which of the plurality of different processing operations are applied by the machine-learned model when processing a given set of input data. The model can be configured to receive and use the seed value(s) to deterministically guide which of the variable processing operations are performed. In such manner, the machine learning model can be used to deterministically perform variable processing of an input. That is, the benefits associated with a machine-learned model having variable processing behavior can be achieved; However, the reproducibility of the model can be guaranteed because the same seed values can later be used to control the model to perform the same processing operation(s), thereby reproducing the same results.

More particularly, models with inherent randomness can offer improved performance by aggregating across multiple model instantiations/runs. One example of models with inherent randomness are models that perform “inference-time-augmentation” (which in some instances can also be referred to as “test-time augmentation”). In inference-time augmentation, a model can be configured to variably perform, over a number of different inference runs, a number of different augmentation operations which mutate the input data to artificially “augment” the number of input data instances. As examples, image augmentation operations can include flipping, rotating, skewing, etc. a single input image to generate one or more augmented input image(s). The results generated during the multiple different inference runs can then be aggregated to provide a single final output per input. Inference-time augmentation has been shown to provide various inference-time benefits, such as, for example, improved accuracy on image classification tasks.

However, inference-time-augmentation and other inference approaches that leverage randomness typically produce slightly different aggregated outputs at each aggregated run when not using a seed value. Thus, the same model may produce a different aggregated output when given the same input. This unpredictable behavior is undesirable in a number of settings (e.g., medical devices or other critical applications) in which reproducibility or consistency are of high importance. For example, a medical device that produces diagnostic predictions on images could predict that an image is positive for a certain diagnosis under one run, yet predict that the same image is negative for the diagnosis on a second run. Note that even small variability in model outputs could result in drastic output changes if this variability is around a specific threshold or operating point of the model.

As a solution to the irreproducibility drawback described above, example implementations of the present disclosure are directed to a machine learning model that is configured and exported in a modified fashion in which the model expects to see and extracts a “seed value” from the given input example. This “seed value” can, in some implementations, be or include one or multiple integers or other variables or values that are uniquely and deterministically set per iteration to allow for a deterministic result. Additionally, in some implementations, the seed value can be deterministically derived from the image or data input itself.

In particular, given one or more models that make use of random deformations or other variable processing to achieve results, those model(s) can be modified to accept a “seed value” held within the input data. In particular, to deterministically control variable elements within the model, one or more seed values can be placed into the input data prior to running inference on the input data. Alternatively, the “seed value” can be derived from the input data itself (e.g., by generating a hash value from the input data). By deterministically passing a particular seed(s) during multiple inference runs, the final results can be made deterministic.

Another example aspect of the present disclosure is directed to the fine-tuning of the specially exported model(s) and/or the seeds selected by customizing or otherwise optimizing the seeds used during inference. For example, with reference to the example of image deformations, one can imagine some deformations providing limited usefulness for inference due to the random nature of the deformation. Typically, such issues can be hidden through performance of a very large number of inference runs averaged together, but this is not an optimal approach as it covers but does not resolve the underlying irreproducibility. Further, a manual evaluation of the usefulness of each possible deformation may be implausible.

The present disclosure provides a solution to the challenge of identifying an improved or optimal set of seed values for use at inference time. In particular, as one example, the following method can be performed to identify key subsets of models that deliver more effective controlled random settings. First, the general approach described herein can be performed to run deterministic seeds on a far larger number of candidate seeds. For example, simulated annealing or similar algorithms can be applied here to identify good seed candidates. Next, subsets of the models and/or seed values that yield superior performance can be identified. For example, this subset can be limited by the number of runs allowable during inference or can be used to reduce the number of runs for increased inference-time efficiency. Once subset(s) of the models have been identified, the seed generation approach can be modified so as to produce optimal seeds on the identified subsets.

The systems and methods of the present disclosure provide a number of technical effects and benefits. As one example technical effect, a system that requires high or absolute reproducibility may have previously not been able to be implemented using models with variable processing such as inference-time augmentation. However, through application of the present disclosure, the benefits of variable processing models (e.g., increased accuracy or other measures of predictive performance) can be extended to systems that require high or absolute reproducibility. Therefore, the techniques described herein represent an improvement in the functionality of a computer itself.

As another example technical effect, the proposed techniques can be used to achieve determinism in any system or method that includes models with variable processing behavior used for the purposes of increased performance. Any system containing one or more models with variable behavior can use this approach to achieve deterministic outputs. For example, models that make use of random inference-time augmentation would need elements of this invention to achieve determinism. Deterministic predictions are highly preferred in medical or other critical applications of machine learning.

Additionally, the proposed method can be used to further improve system performance prior to deployment or testing by establishing subsets of models and/or seeds for superior performance. Thus, the seed values described herein represent an additional opportunity to perform model optimization for different deployment contexts.

Finally, the present disclosure provides a more efficient technique to achieve deterministic results in a variable processing context. Specifically, one alternative approach may be to create multiple different models that each have deterministic behavior on their own, but which provide different processing behavior relative to each other. This alternative approach may achieve the benefits of having multiple processing behaviors in a deterministic fashion, but is significantly less efficient because it requires the creation, training, storage, and deployment of multiple different models. The techniques described herein in which a model having variable behavior can be deterministically controlled using seed values therefore represent an improvement in computer efficiency, such as reduced processor usage, reduced memory usage, reduced usage of network bandwidth, etc.

With reference now to the Figures, example embodiments of the present disclosure will be discussed in further detail.

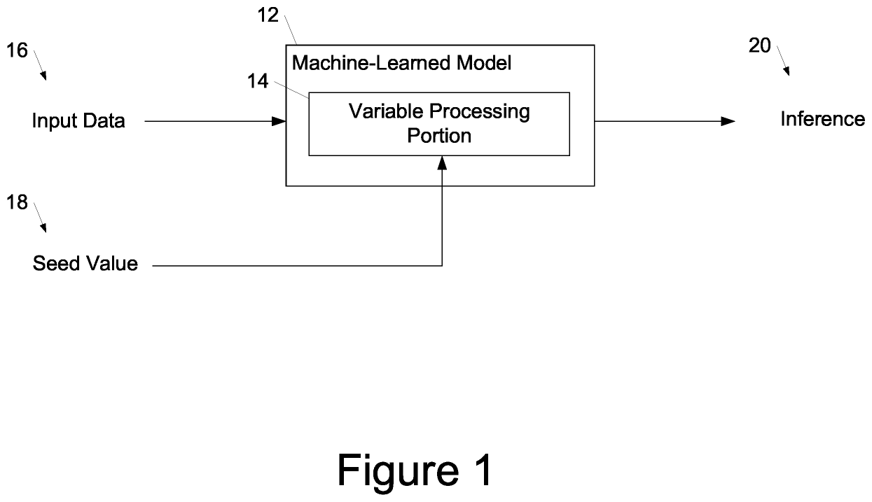

FIG. 1 depicts a block diagram of example techniques to perform deterministic inference for a machine-learned model 12 with variable behavior according to example embodiments of the present disclosure.

According to an aspect of the present disclosure, the machine-learned model 12 includes a variable processing portion 14 that is configured to variably apply one or more of a plurality of different processing operations when processing a set of input data 16 to produce an inference 20.

Specifically, the variable processing portion 14 can be configured to receive a seed value 18 alongside, as part of, or otherwise in association with the input data 16. The seed value 18 can deterministically control which of the variable processing operations are performed by the variable processing portion 14 of the machine-learned model 12.

There are many different types of input data 16 that the machine learned model 12 can process, including:

Numeric data: Numeric data can include numerical values that can be used as input to a machine learning model. This includes data such as integers, floating point numbers, and statistical measures like mean and standard deviation.

Categorical data: Categorical data can include discrete values that can be used to classify data points into categories. This includes data such as names, labels, or categories like “red” or “blue.”

Time series data: Time series data can include sequential data points collected over time. This can include data such as inventory data, weather measurements, traffic patterns, or medical images of the same patient over the course of time.

Text data: Text data can include words or other written language that can be used as input to a machine learning model. This can include data such as emails, social media posts, or news articles.

Image data: Image data can include pixel values that represent an image. This can include data such as photographs, video frames, or medical scans.

Other types of input data 16 can be used additionally or alternatively.

The inference 20 can be any type of inference. Some example inference types include:

Classification inferences: In classification, a model is trained to predict the class or category of a given input data point. For example, a model might be trained to classify emails as spam or not spam, or to classify images as pictures of cats or dogs.

Regression inferences: In regression, a model is trained to predict a continuous value, such as a price or a probability. For example, a model might be trained to predict the price of a house based on its size, location, and other features.

Clustering inferences: In clustering, a model is trained to group data points into clusters based on their similarity.

Anomaly detection inferences: In anomaly detection, a model is trained to identify unusual or unexpected data points within a dataset. This can be useful for detecting fraud, for example, or for identifying equipment failures in a manufacturing process.

Other types of inferences 20 can be produced additionally or alternatively.

As indicated above, the machine-learned model 12 can be configured to perform variable processing operations. The model 12 can include a variable processing portion 14 that is capable of performing a variety of different types of processing, including but not limited to data processing, feature extraction, and classification. The model 12 can be configured to receive a seed value 18 that controls which type of processing should be performed by the variable processing portion 14.

The variable processing portion 14 is configured to dynamically adjust its behavior based on the seed value 18, allowing the model 12 to deterministically adapt to different processing behaviors as instructed.

The seed value 18 can be a number (e.g., integer, floating point number, etc.) or other input value(s) used to deterministically control the variable processing portion 14.

The seed value 18 can be used to ensure that the results of an otherwise random process are reproducible.

There are several different types of processing operations performed by the variable processing portion 14 of the machine-learned model 12 that can be varied (e.g., deterministically controlled) during inference. Some example variable processing behaviors are described in the following paragraphs.

In some implementations, the variable processing operations performable by the variable processing portion 14 correspond to variable data preprocessing operations: The model 12 may preprocess the input data 16 before making the inference 20. Preprocessing can include tasks such as imputing missing values, scaling or normalizing numerical features, and encoding categorical variables. Whether the model 12 performs these operations at all or with different parameterizations can be varied.

In some implementations, the variable data preprocessing operations correspond to variable data augmentation operations. Some possible data augmentation strategies include:

Random cropping: This involves randomly selecting a subset of the original image and using it as a new data sample.

Random flipping: This involves randomly flipping the original image horizontally or vertically and using the flipped version as a new data sample. More generally, input data with a number of dimensions (e.g., 1, 2, more than two, etc.) can be “flipped” or rotated along any of the dimensions or axes.

Random rotation: This involves randomly rotating the original image by a certain angle and using the rotated version as a new data sample. More generally, an orthonormal transformation can be perfomed on input data with respect to any of the dimensions or axes of the input data.

Random deformations: Application of a randomly generated deformation field to the given image or other input.

Color jitter: This involves randomly altering the color channels of the original image, such as by adding noise or changing the brightness or contrast.

Adding noise: This involves adding noise to the original image, such as by adding Gaussian noise or salt and pepper noise. These are just a few examples of data augmentation strategies that can be used. There are many other possible strategies as well.

In some implementations, performing some or all of the variable data augmentation operations described above can result in a model that performs (e.g., in a deterministic fashion) a inference-time augmentation (ITA) approach. ITA is a technique in which multiple versions of a inputsample are generated and fed into the machine learning model, with the results from each version being aggregated to produce a final prediction. The use of ITA can help to improve the performance of the machine learning model, as it allows the model to make more robust and accurate predictions. This can be especially useful in cases where the input sample may be noisy or have other types of variations that could affect the model's performance.

In some implementations, the variable processing operations performable by the variable processing portion 14 correspond to usages of different submodel(s) or model(s) of an ensemble: In some implementations, the model 12 may include a number of different submodels or individual models within an ensemble. Depending on the application, the variable processing portion 14 can correspond to a different subset of such submodels or individual model(s) that are used for a particular inference run.

More particularly, in some implementations, the model 12 can be an ensemble of models: Instead of using a single model for inference, an ensemble of multiple models can be used to generate inferences. Therefore, in some implementations, the seed value 18 can control or select which one or more of a plurality of models in the ensemble are used for a given inference run.

There are many different techniques to generate model ensembles, including: Boosting: This involves training a sequence of weak models and combining them to form a strong model. The weak models are trained sequentially, with each model attempting to correct the errors of the previous model. Bagging: This involves training multiple models in parallel and combining their predictions using a simple averaging or majority vote. Stacking: This involves training multiple models and using a meta-model to combine their predictions. The meta-model is trained using the predictions of the individual models as input features. Random forests: This is a type of model ensemble that involves training a large number of decision trees and combining their predictions using a majority vote.

In some implementations, the variable processing operations performable by the variable processing portion 14 correspond to a dynamically configurable set of parameters, depth, or width for or of the model 12. For example, the variable processing portion 14 can have a reconfigurable or variable set of parameters, depth, or width. Which of the variable parameters, depth, or width are used can be controlled by the seed value 18. The ability to dynamically adjust the model's parameters, depth, or width allows the model 12 to adapt to different tasks and data sets, improving its flexibility and versatility. This can result in improved performance on a wide range of tasks and datasets.

In machine learning, the parameters of a model refer to the values that are learned and adjusted during the training process. These parameters are used to control the behavior of the model and to make predictions about data. There are different types of parameters that can be used in a machine learning model, depending on the type of model and the problem it is being used to solve. Some common types of parameters include: Weights: In many machine learning models, weights are used to control the strength or importance of different features or inputs. For example, in a neural network, weights are used to control the relative contribution of each input to the output of the model. Biases: Biases are used in some machine learning models to shift the output of the model. For example, in a linear regression model, a bias term is used to adjust the predicted output value. Hyperparameters: Hyperparameters are parameters that are set by the user or researcher, rather than being learned from the data. They control the overall behavior and complexity of the model, and can include things like the learning rate, the number of hidden layers in a neural network, or the degree of a polynomial in a polynomial regression model. Latent variables: In some types of models, such as latent variable models, parameters are used to represent hidden or unobserved variables that are believed to influence the observed data. By adjusting the values of these and other parameters, it is possible to enable variable processing behavior by the machine-learned model 12.

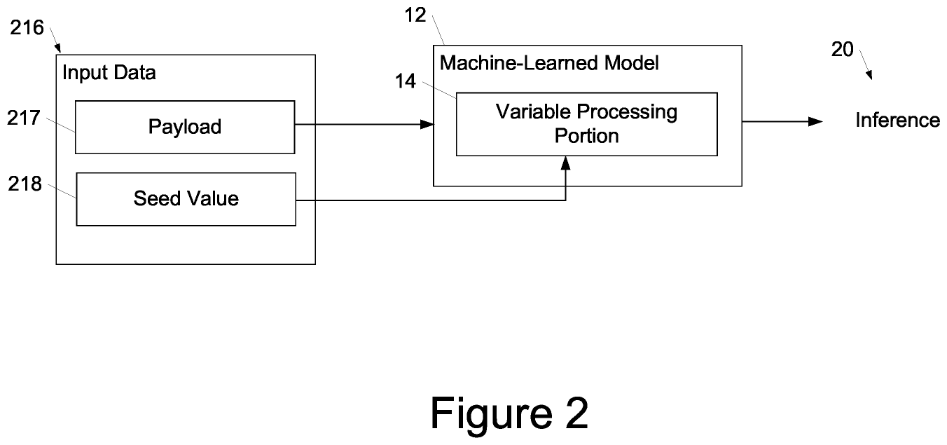

In some implementations, the seed value 18 can be added into or otherwise form a part of the input data 16. For example, FIG. 2 shows an example approach in which a set of input data 216 contains a payload 217 and a seed value 218. For example, the payload 217 can refer to the portion of the input data 216 on which the inference 20 is to be generated. For example, the payload 217 may correspond to an image such as a medical image (e.g., x-ray, CT scan, etc.). Thus, in some implementations, the model 12 is configured to receive the seed value 218 as a part of the input data 216.

In some implementations, the seed value 218 can be stored with the payload 217 in a same data file. Thereafter, whenever inference is performed with respect to the payload 217, the model 12 can deterministically generate the same inference 20. As examples, the seed value 218 can be stored as metadata for the payload 217; the seed value 218 can be stored in sequence or encoded as a single data stream with the payload 217; the seed value 218 can be added as a watermark to the payload 217; the seed value 218 can be derived from the payload (e.g., as a hash-value derived from the image); and/or other forms of logical association between the seed value 218 and the payload 217 to form a set of input data 216.

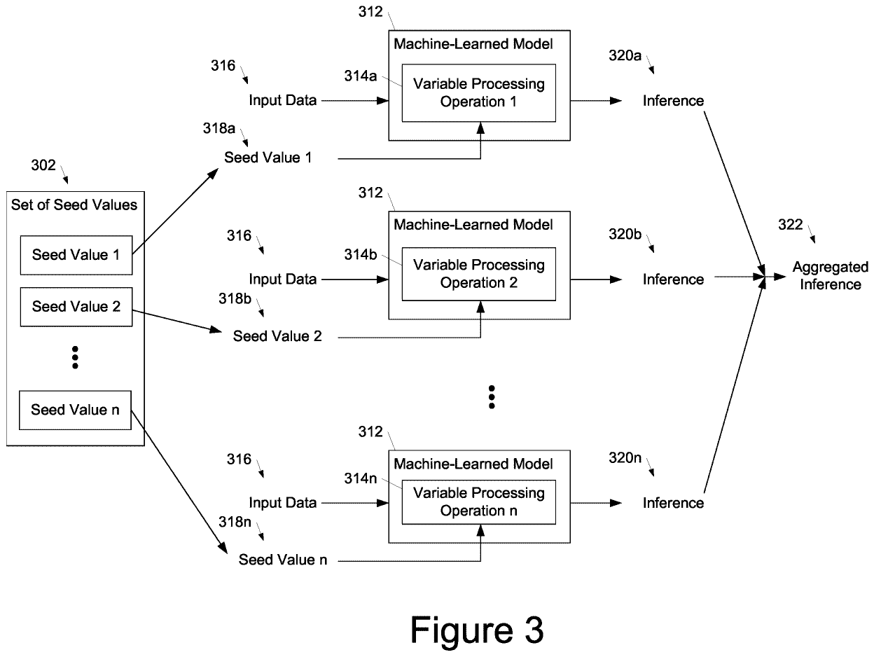

As described above, in some implementations, the variable processing behavior of the machine-learned model can be used to perform multiple different processing operations on an input to generate multiple different intermediate inferences and then an aggregate inference can be produced from the multiple different intermediate inferences. FIG. 3 shows one example of this approach.

Specifically, FIG. 3 illustrates a scenario in which a machine-learned model 312 performs varied, but deterministic processing behavior over multiple inference runs to generate an aggregated inference 322 for a set of input data 316. In particular, a number (n) of inference runs can be performed. At each inference run, the machine-learned model 312 can receive one of a set of seed values 302. For example, at inference run 1, the model 312 can receive seed value 1318a, which causes the model 312 to perform variable processing operations 1314a while processing the input data 316 to generate an intermediate inference 320a. For example, at inference run 2, the model 312 can receive seed value 2318b, which causes the model 312 to perform variable processing operations 2314b while processing the input data 316 to generate an intermediate inference 320b. For example, at inference run n, the model 312 can receive seed value n 318n, which causes the model 312 to perform variable processing operations n 314n while processing the input data 316 to generate an intermediate inference 320n. In some implementations, a single seed value can determine multiple processing operations.

The intermediate inferences 310a-n can be aggregated to generate the aggregated inference 322. To generate the aggregated inference 322 (which can also be referred to as the “ensemble” output) various forms of aggregation can optionally be performed. Some possible methods of aggregating intermediate inferences include:

Majority voting: In majority voting, the final inference is determined by the majority of intermediate inferences. For example, if a model makes three intermediate inferences, and two of them predict one outcome and one predicts another, the majority vote would be for the outcome predicted by the two intermediate inferences.

Weighted voting: In weighted voting, each intermediate inference is assigned a weight, and the final inference is determined based on the weighted average of the intermediate inferences. This can be useful if some intermediate inferences are considered more reliable or accurate than others.

Averaging: In averaging, the final inference is determined by taking the mean of the intermediate inferences. This can be useful if the intermediate inferences are expected to be equally reliable and accurate.

Stacking: In stacking, a second machine learning model is trained to make a final inference based on the intermediate inferences made by the first model. This can be useful if the intermediate inferences contain valuable information that is not captured by the final inference made by the first model.

By aggregating or combining the multiple intermediate inferences 320a-n in these or other ways, it is possible to generate the aggregated inference 322 that is typically more accurate or reliable than any individual intermediate inference.

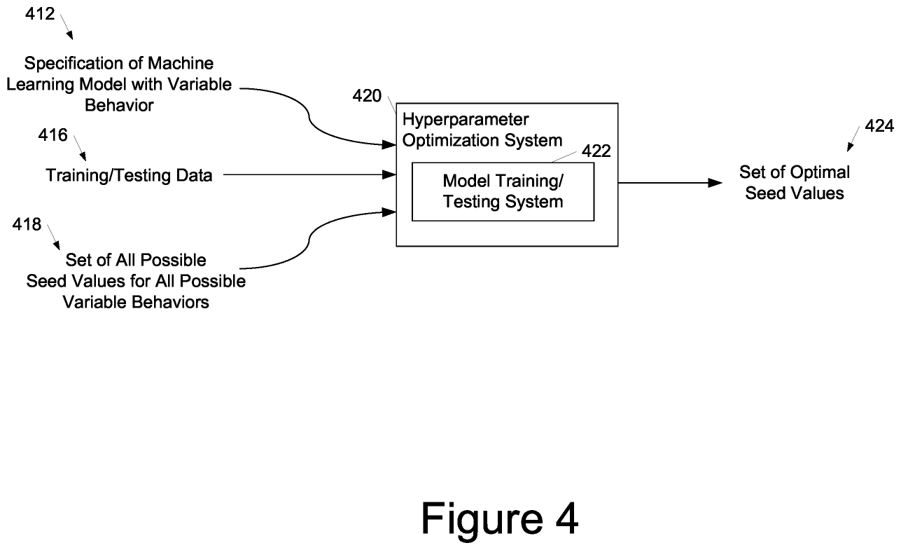

Another example aspect of the present disclosure is directed to identifying an improved or optimal set of seed values for use at inference time. As one example, FIG. 4 depicts a block diagram of an example technique to determine a set of optimal seed values 424 according to example embodiments of the present disclosure. In particular, the present disclosure proposes to determine the set of optimal seed values 424 by treating them as hyperparameters of the model and identifying them via a hyperparameter optimization process.

Specifically, as illustrated in FIG. 4, a hyperparameter optimization system 420 can receive: a specification 412 of the machine learning model with variable behavior (e.g., a TensorFlow graph of similar); training and/or testing data 416; and (in some implementations) a set 418 of possible seed values for possible variable behaviors. The set of seed values 418 may in some cases be an actual set of values while in other cases it may be a specification or definition of acceptable value ranges within a search space or other description of possible seed values or parameterization of seed values.

The hyperparameter optimization system 420 can perform (e.g., using a model training/testing system 422) a hyperparameter optimization technique to determine the set of optimal seed values 424 that result in optimal performance of the machine learning model specified by the specification 412, as measured relative to the training and/or testing data 416.

In particular, the hyperparameter optimization technique can be or include a number of different techniques. Some example hyperparameter optimization techniques include:

Grid search: This is a brute-force approach that involves training the model with a range of different hyperparameter values and selecting the best combination of values based on the performance of the model.

Random search: This involves randomly sampling from the space of possible hyperparameter values and selecting the best combination of values based on the performance of the model.

Bayesian optimization: This is a probabilistic approach that uses a surrogate model to approximate the optimization landscape and iteratively selects the next set of hyperparameters to evaluate based on the current model performance.

Genetic algorithms: This is a metaheuristic optimization algorithm that is inspired by natural selection and genetics. It involves creating a population of candidate solutions, evaluating their performance, and iteratively evolving the population through selection, crossover, and mutation operations.

Gradient-based optimization: This is an optimization method that uses the gradient of the objective function to guide the search for the optimal hyperparameter values.

Hyperband: This is a resource-aware algorithm that uses the resource-accuracy tradeoff to quickly identify promising hyperparameters. It works by training models with different budgets (e.g., number of epochs, number of training examples) and identifying the best performing model at each budget. It then uses this information to guide the search for the optimal hyperparameters.

Simulated Annealing: Simulated annealing is a metaheuristic optimization algorithm that can be used to find the global minimum of a function by iteratively searching for the optimal solution using a probabilistic search process. The algorithm works by randomly sampling from the space of possible solutions and accepting or rejecting the proposed solution based on its quality and the current temperature of the system. At high temperatures, the algorithm is more likely to accept solutions that are worse than the current solution, allowing it to explore a wider range of possibilities. As the temperature decreases, the algorithm becomes more selective and is less likely to accept worse solutions, allowing it to converge towards the optimal solution.

Simulated annealing is often used for optimization problems that have many local minima, as it is able to escape from these local minima and explore the global search space. It is also useful for problems with noisy or complex objective functions, as it is able to tolerate a certain amount of noise and still find the optimal solution.

The choice of hyperparameter optimization algorithm can depend on the specific characteristics of the machine learning model and the optimization problem, as well as the available computational resources.

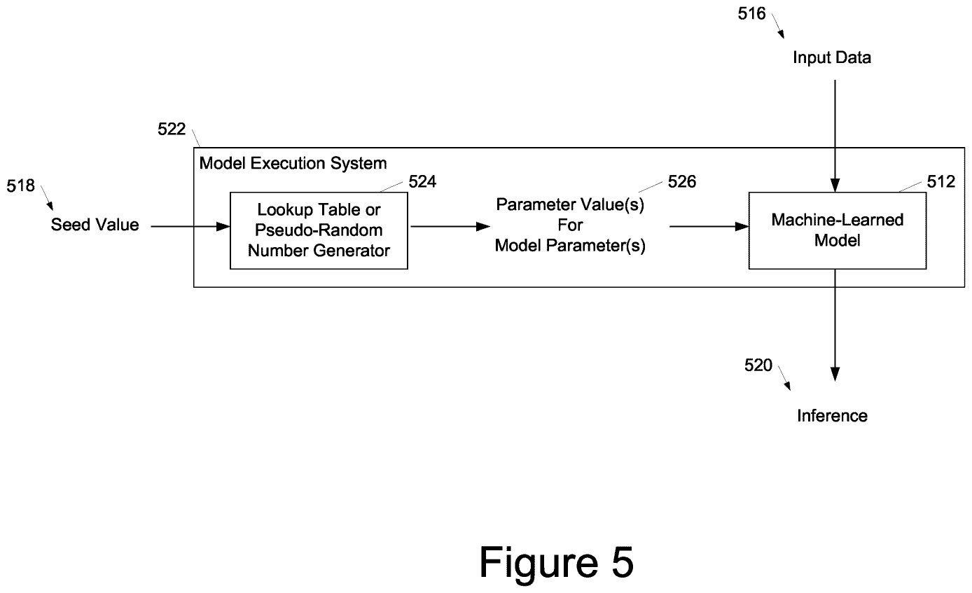

FIG. 5 depicts a block diagram of an example technique to implement deterministic inference for a machine-learned model 512 with variable behavior according to example embodiments of the present disclosure. In particular, FIG. 5 illustrates a model execution system 522.

The model execution system 522 can receive a seed value 518. The model execution system 522 can use a lookup table 524 or a pseudo-random number generator 524 to generate parameter value(s) 526 for model parameter(s) of the machine-learned model 512. The parameter value(s) can be values that, when inserted into the machine-learned model 512, cause the model 512 to deterministically perform some subset of possible variable processing operations performable by the model 512.

Once the parameter value(s) 526 are loaded into the model 512, the model 512 can process a set of input data 516 to generate an inference 520.

Thus, the example system 522 uses a lookup table or a pseudo-random number generator to convert from a seed value 518 to specific model parameter value(s) 526.

A lookup table, also known as a “table lookup” or “static table,” is a data structure that allows for efficient storage and retrieval of data based on input seed values. Lookup tables can be used to store data that is used frequently or that needs to be accessed quickly. Lookup tables can be implemented in various ways, such as using an array or a hash table, and can be used in conjunction with algorithms that require fast access to data. One advantage of lookup tables is that they can allow for fast access to data, as long as the seed used to search for the corresponding parameter value(s) is known.

A random number generator is a mathematical function or algorithm that generates a sequence of random numbers. These numbers are usually generated using a seed value, which is used to initialize the generator and determine the sequence of numbers that will be produced. There are many different algorithms and techniques for generating random numbers, and the choice of generator will depend on the specific requirements and constraints of the application. Some common types of random number generators include:

Pseudorandom number generators (PRNGs): These are algorithms that use a mathematical formula to produce a sequence of numbers that appear to be random, but are actually deterministic.

Hybrid random number generators: These are generators that use a combination of PRNGs and true random number generators (TRNGs) to produce random numbers. TRNGs are physical devices that use external sources of randomness, such as noise in electronic circuits or radioactive decay, to generate genuinely random numbers.

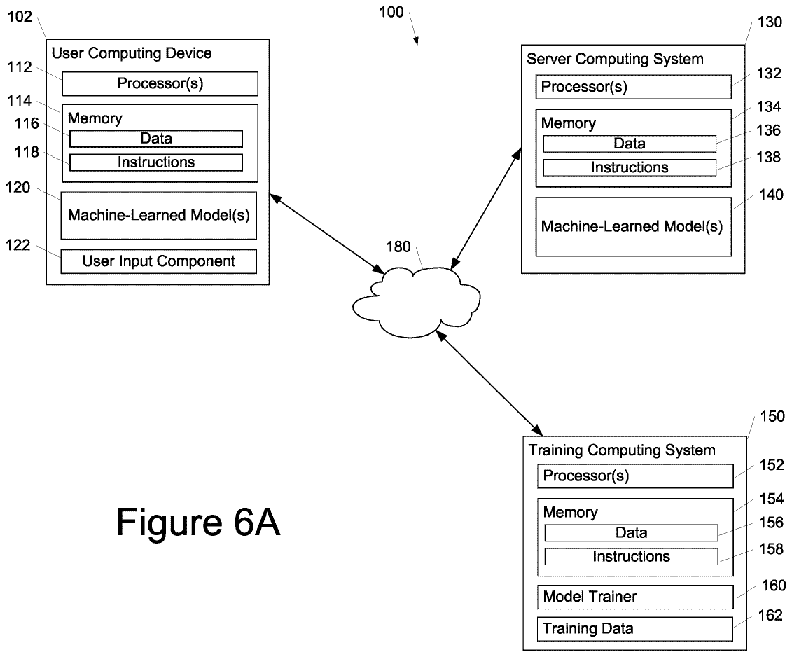

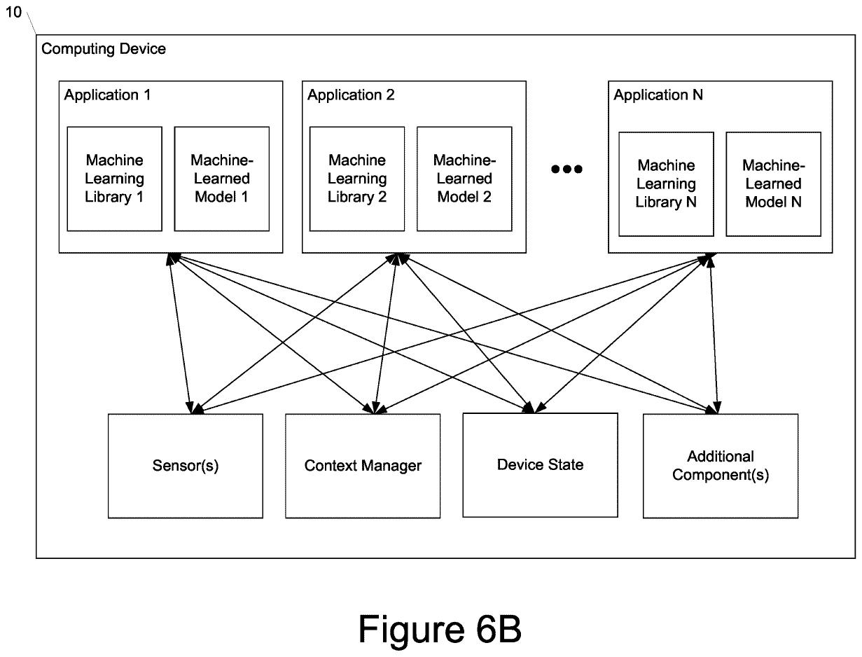

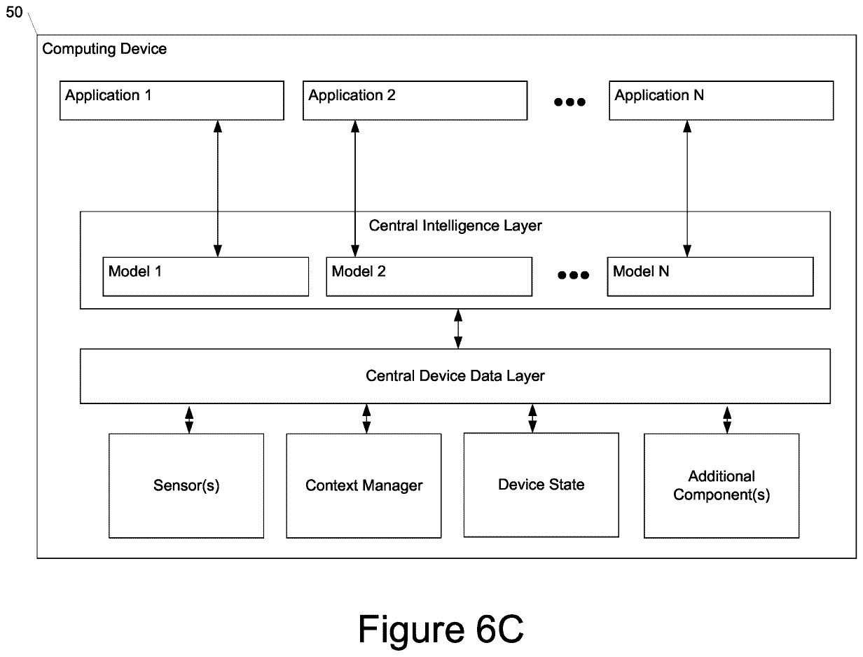

FIG. 6A depicts a block diagram of an example computing system 100 according to example embodiments of the present disclosure. The system 100 includes a user computing device 102, a server computing system 130, and a training computing system 150 that are communicatively coupled over a network 180.

The user computing device 102 can be any type of computing device, such as, for example, a personal computing device (e.g., laptop or desktop), a mobile computing device (e.g., smartphone or tablet), a gaming console or controller, a wearable computing device, an embedded computing device, or any other type of computing device.

The user computing device 102 includes one or more processors 112 and a memory 114. The one or more processors 112 can be any suitable processing device (e.g., a processor core, a microprocessor, an ASIC, an FPGA, a controller, a microcontroller, etc.) and can be one processor or a plurality of processors that are operatively connected. The memory 114 can include one or more non-transitory computer-readable storage media, such as RAM, ROM, EEPROM, EPROM, flash memory devices, magnetic disks, etc., and combinations thereof. The memory 114 can store data 116 and instructions 118 which are executed by the processor 112 to cause the user computing device 102 to perform operations.

In some implementations, the user computing device 102 can store or include one or more machine-learned models 120. For example, the machine-learned models 120 can be or can otherwise include various machine-learned models such as neural networks (e.g., deep neural networks) or other types of machine-learned models, including non-linear models and/or linear models. Neural networks can include feed-forward neural networks, recurrent neural networks (e.g., long short-term memory recurrent neural networks), convolutional neural networks or other forms of neural networks. Some example machine-learned models can leverage an attention mechanism such as self-attention. For example, some example machine-learned models can include multi-headed self-attention models (e.g., transformer models). Example machine-learned models 120 are discussed with reference to FIGS. 1-5.

In some implementations, the one or more machine-learned models 120 can be received from the server computing system 130 over network 180, stored in the user computing device memory 114, and then used or otherwise implemented by the one or more processors 112. In some implementations, the user computing device 102 can implement multiple parallel instances of a single machine-learned model 120 (e.g., to perform parallel variable processing across the same input multiple instances of inputs).

Additionally or alternatively, one or more machine-learned models 140 can be included in or otherwise stored and implemented by the server computing system 130 that communicates with the user computing device 102 according to a client-server relationship. For example, the machine-learned models 140 can be implemented by the server computing system 140 as a portion of a web service. Thus, one or more models 120 can be stored and implemented at the user computing device 102 and/or one or more models 140 can be stored and implemented at the server computing system 130. For example, the user computing device 102 and/or the server computing system 130 can include and/or implement the model execution system 522 shown in FIG. 5.

The user computing device 102 can also include one or more user input components 122 that receives user input. For example, the user input component 122 can be a touch-sensitive component (e.g., a touch-sensitive display screen or a touch pad) that is sensitive to the touch of a user input object (e.g., a finger or a stylus). The touch-sensitive component can serve to implement a virtual keyboard. Other example user input components include a microphone, a traditional keyboard, or other means by which a user can provide user input.

The server computing system 130 includes one or more processors 132 and a memory 134. The one or more processors 132 can be any suitable processing device (e.g., a processor core, a microprocessor, an ASIC, an FPGA, a controller, a microcontroller, etc.) and can be one processor or a plurality of processors that are operatively connected. The memory 134 can include one or more non-transitory computer-readable storage media, such as RAM, ROM, EEPROM, EPROM, flash memory devices, magnetic disks, etc., and combinations thereof. The memory 134 can store data 136 and instructions 138 which are executed by the processor 132 to cause the server computing system 130 to perform operations.

In some implementations, the server computing system 130 includes or is otherwise implemented by one or more server computing devices. In instances in which the server computing system 130 includes plural server computing devices, such server computing devices can operate according to sequential computing architectures, parallel computing architectures, or some combination thereof.

As described above, the server computing system 130 can store or otherwise include one or more machine-learned models 140. For example, the models 140 can be or can otherwise include various machine-learned models. Example machine-learned models include neural networks or other multi-layer non-linear models. Example neural networks include feed forward neural networks, deep neural networks, recurrent neural networks, and convolutional neural networks. Some example machine-learned models can leverage an attention mechanism such as self-attention. For example, some example machine-learned models can include multi-headed self-attention models (e.g., transformer models). Example models 140 are discussed with reference to FIGS. 1-5.

The user computing device 102 and/or the server computing system 130 can train the models 120 and/or 140 via interaction with the training computing system 150 that is communicatively coupled over the network 180. The training computing system 150 can be separate from the server computing system 130 or can be a portion of the server computing system 130.

The training computing system 150 includes one or more processors 152 and a memory 154. The one or more processors 152 can be any suitable processing device (e.g., a processor core, a microprocessor, an ASIC, an FPGA, a controller, a microcontroller, etc.) and can be one processor or a plurality of processors that are operatively connected. The memory 154 can include one or more non-transitory computer-readable storage media, such as RAM, ROM, EEPROM, EPROM, flash memory devices, magnetic disks, etc., and combinations thereof. The memory 154 can store data 156 and instructions 158 which are executed by the processor 152 to cause the training computing system 150 to perform operations. In some implementations, the training computing system 150 includes or is otherwise implemented by one or more server computing devices.

The training computing system 150 can include a model trainer 160 that trains the machine-learned models 120 and/or 140 stored at the user computing device 102 and/or the server computing system 130 using various training or learning techniques, such as, for example, backwards propagation of errors. For example, a loss function can be backpropagated through the model(s) to update one or more parameters of the model(s) (e.g., based on a gradient of the loss function). Various loss functions can be used such as mean squared error, likelihood loss, cross entropy loss, hinge loss, and/or various other loss functions. Gradient descent techniques can be used to iteratively update the parameters over a number of training iterations.

In some implementations, performing backwards propagation of errors can include performing truncated backpropagation through time. The model trainer 160 can perform a number of generalization techniques (e.g., weight decays, dropouts, etc.) to improve the generalization capability of the models being trained.

In particular, the model trainer 160 can train the machine-learned models 120 and/or 140 based on a set of training data 162. In some implementations, if the user has provided consent, the training examples can be provided by the user computing device 102. Thus, in such implementations, the model 120 provided to the user computing device 102 can be trained by the training computing system 150 on user-specific data received from the user computing device 102. In some instances, this process can be referred to as personalizing the model. In some implementations, the model trainer 160 can include and/or implement the hyperparameter optimization system 420 of FIG. 4.

The model trainer 160 includes computer logic utilized to provide desired functionality. The model trainer 160 can be implemented in hardware, firmware, and/or software controlling a general purpose processor. For example, in some implementations, the model trainer 160 includes program files stored on a storage device, loaded into a memory and executed by one or more processors. In other implementations, the model trainer 160 includes one or more sets of computer-executable instructions that are stored in a tangible computer-readable storage medium such as RAM, hard disk, or optical or magnetic media.

The network 180 can be any type of communications network, such as a local area network (e.g., intranet), wide area network (e.g., Internet), or some combination thereof and can include any number of wired or wireless links. In general, communication over the network 180 can be carried via any type of wired and/or wireless connection, using a wide variety of communication protocols (e.g., TCP/IP, HTTP, SMTP, FTP), encodings or formats (e.g., HTML, XML), and/or protection schemes (e.g., VPN, secure HTTP, SSL).

The machine-learned models described in this specification may be used in a variety of tasks, applications, and/or use cases.

In some implementations, the input to the machine-learned model(s) of the present disclosure can be image data. The machine-learned model(s) can process the image data to generate an output. As an example, the machine-learned model(s) can process the image data to generate an image recognition output (e.g., a recognition of the image data, a latent embedding of the image data, an encoded representation of the image data, a hash of the image data, etc.). As another example, the machine-learned model(s) can process the image data to generate an image segmentation output. As another example, the machine-learned model(s) can process the image data to generate an image classification output. As another example, the machine-learned model(s) can process the image data to generate an image data modification output (e.g., an alteration of the image data, etc.). As another example, the machine-learned model(s) can process the image data to generate an encoded image data output (e.g., an encoded and/or compressed representation of the image data, etc.). As another example, the machine-learned model(s) can process the image data to generate an upscaled image data output. As another example, the machine-learned model(s) can process the image data to generate a prediction output.

In some implementations, the input to the machine-learned model(s) of the present disclosure can be text or natural language data. The machine-learned model(s) can process the text or natural language data to generate an output. As an example, the machine-learned model(s) can process the natural language data to generate a language encoding output. As another example, the machine-learned model(s) can process the text or natural language data to generate a latent text embedding output. As another example, the machine-learned model(s) can process the text or natural language data to generate a translation output. As another example, the machine-learned model(s) can process the text or natural language data to generate a classification output. As another example, the machine-learned model(s) can process the text or natural language data to generate a textual segmentation output. As another example, the machine-learned model(s) can process the text or natural language data to generate a semantic intent output. As another example, the machine-learned model(s) can process the text or natural language data to generate an upscaled text or natural language output (e.g., text or natural language data that is higher quality than the input text or natural language, etc.). As another example, the machine-learned model(s) can process the text or natural language data to generate a prediction output.

In some implementations, the input to the machine-learned model(s) of the present disclosure can be speech data. The machine-learned model(s) can process the speech data to generate an output. As an example, the machine-learned model(s) can process the speech data to generate a speech recognition output. As another example, the machine-learned model(s) can process the speech data to generate a speech translation output. As another example, the machine-learned model(s) can process the speech data to generate a latent embedding output. As another example, the machine-learned model(s) can process the speech data to generate an encoded speech output (e.g., an encoded and/or compressed representation of the speech data, etc.). As another example, the machine-learned model(s) can process the speech data to generate an upscaled speech output (e.g., speech data that is higher quality than the input speech data, etc.). As another example, the machine-learned model(s) can process the speech data to generate a textual representation output (e.g., a textual representation of the input speech data, etc.). As another example, the machine-learned model(s) can process the speech data to generate a prediction output.

In some implementations, the input to the machine-learned model(s) of the present disclosure can be latent encoding data (e.g., a latent space representation of an input, etc.). The machine-learned model(s) can process the latent encoding data to generate an output. As an example, the machine-learned model(s) can process the latent encoding data to generate a recognition output. As another example, the machine-learned model(s) can process the latent encoding data to generate a reconstruction output. As another example, the machine-learned model(s) can process the latent encoding data to generate a search output. As another example, the machine-learned model(s) can process the latent encoding data to generate a reclustering output. As another example, the machine-learned model(s) can process the latent encoding data to generate a prediction output.

In some implementations, the input to the machine-learned model(s) of the present disclosure can be statistical data. Statistical data can be, represent, or otherwise include data computed and/or calculated from some other data source. The machine-learned model(s) can process the statistical data to generate an output. As an example, the machine-learned model(s) can process the statistical data to generate a recognition output. As another example, the machine-learned model(s) can process the statistical data to generate a prediction output. As another example, the machine-learned model(s) can process the statistical data to generate a classification output. As another example, the machine-learned model(s) can process the statistical data to generate a segmentation output. As another example, the machine-learned model(s) can process the statistical data to generate a visualization output. As another example, the machine-learned model(s) can process the statistical data to generate a diagnostic output.

In some implementations, the input to the machine-learned model(s) of the present disclosure can be sensor data. The machine-learned model(s) can process the sensor data to generate an output. As an example, the machine-learned model(s) can process the sensor data to generate a recognition output. As another example, the machine-learned model(s) can process the sensor data to generate a prediction output. As another example, the machine-learned model(s) can process the sensor data to generate a classification output. As another example, the machine-learned model(s) can process the sensor data to generate a segmentation output. As another example, the machine-learned model(s) can process the sensor data to generate a visualization output. As another example, the machine-learned model(s) can process the sensor data to generate a diagnostic output. As another example, the machine-learned model(s) can process the sensor data to generate a detection output.

In some cases, the machine-learned model(s) can be configured to perform a task that includes encoding input data for reliable and/or efficient transmission or storage (and/or corresponding decoding). For example, the task may be an audio compression task. The input may include audio data and the output may comprise compressed audio data. In another example, the input includes visual data (e.g. one or more images or videos), the output comprises compressed visual data, and the task is a visual data compression task. In another example, the task may comprise generating an embedding for input data (e.g. input audio or visual data).

In some cases, the input includes visual data and the task is a computer vision task. In some cases, the input includes pixel data for one or more images and the task is an image processing task. For example, the image processing task can be image classification, where the output is a set of scores, each score corresponding to a different object class and representing the likelihood that the one or more images depict an object belonging to the object class. The image processing task may be object detection, where the image processing output identifies one or more regions in the one or more images and, for each region, a likelihood that region depicts an object of interest. As another example, the image processing task can be image segmentation, where the image processing output defines, for each pixel in the one or more images, a respective likelihood for each category in a predetermined set of categories. For example, the set of categories can be foreground and background. As another example, the set of categories can be object classes. As another example, the image processing task can be depth estimation, where the image processing output defines, for each pixel in the one or more images, a respective depth value. As another example, the image processing task can be motion estimation, where the network input includes multiple images, and the image processing output defines, for each pixel of one of the input images, a motion of the scene depicted at the pixel between the images in the network input.

In some cases, the input includes audio data representing a spoken utterance and the task is a speech recognition task. The output may comprise a text output which is mapped to the spoken utterance. In some cases, the task comprises encrypting or decrypting input data. In some cases, the task comprises a microprocessor performance task, such as branch prediction or memory address translation.

FIG. 6A illustrates one example computing system that can be used to implement the present disclosure. Other computing systems can be used as well. For example, in some implementations, the user computing device 102 can include the model trainer 160 and the training dataset 162. In such implementations, the models 120 can be both trained and used locally at the user computing device 102. In some of such implementations, the user computing device 102 can implement the model trainer 160 to personalize the models 120 based on user-specific data.

FIG. 6B depicts a block diagram of an example computing device 10 that performs according to example embodiments of the present disclosure. The computing device 10 can be a user computing device or a server computing device.

The computing device 10 includes a number of applications (e.g., applications 1 through N). Each application contains its own machine learning library and machine-learned model(s). For example, each application can include a machine-learned model. Example applications include a text messaging application, an email application, a dictation application, a virtual keyboard application, a browser application, etc.

As illustrated in FIG. 6B, each application can communicate with a number of other components of the computing device, such as, for example, one or more sensors, a context manager, a device state component, and/or additional components. In some implementations, each application can communicate with each device component using an API (e.g., a public API). In some implementations, the API used by each application is specific to that application.

FIG. 6C depicts a block diagram of an example computing device 50 that performs according to example embodiments of the present disclosure. The computing device 50 can be a user computing device or a server computing device.

The computing device 50 includes a number of applications (e.g., applications 1 through N). Each application is in communication with a central intelligence layer. Example applications include a text messaging application, an email application, a dictation application, a virtual keyboard application, a browser application, etc. In some implementations, each application can communicate with the central intelligence layer (and model(s) stored therein) using an API (e.g., a common API across all applications).

The central intelligence layer includes a number of machine-learned models. For example, as illustrated in FIG. 6C, a respective machine-learned model can be provided for each application and managed by the central intelligence layer. In other implementations, two or more applications can share a single machine-learned model. For example, in some implementations, the central intelligence layer can provide a single model for all of the applications. In some implementations, the central intelligence layer is included within or otherwise implemented by an operating system of the computing device 50.

The central intelligence layer can communicate with a central device data layer. The central device data layer can be a centralized repository of data for the computing device 50. As illustrated in FIG. 6C, the central device data layer can communicate with a number of other components of the computing device, such as, for example, one or more sensors, a context manager, a device state component, and/or additional components. In some implementations, the central device data layer can communicate with each device component using an API (e.g., a private API).

The technology discussed herein makes reference to servers, databases, software applications, and other computer-based systems, as well as actions taken and information sent to and from such systems. The inherent flexibility of computer-based systems allows for a great variety of possible configurations, combinations, and divisions of tasks and functionality between and among components. For instance, processes discussed herein can be implemented using a single device or component or multiple devices or components working in combination. Databases and applications can be implemented on a single system or distributed across multiple systems. Distributed components can operate sequentially or in parallel.

While the present subject matter has been described in detail with respect to various specific example embodiments thereof, each example is provided by way of explanation, not limitation of the disclosure. Those skilled in the art, upon attaining an understanding of the foregoing, can readily produce alterations to, variations of, and equivalents to such embodiments. Accordingly, the subject disclosure does not preclude inclusion of such modifications, variations and/or additions to the present subject matter as would be readily apparent to one of ordinary skill in the art. For instance, features illustrated or described as part of one embodiment can be used with another embodiment to yield a still further embodiment. Thus, it is intended that the present disclosure cover such alterations, variations, and equivalents.

1. A computer system that performs deterministic inference for a machine-learned model with variable behavior, the computer system comprising:
one or more processors; and
one or more non-transitory computer-readable media that collectively store:
the machine-learned model, wherein the machine-learned model is configured to variably apply one or more of a plurality of different processing operations when processing an input; and
instructions that, when executed by the one or more processors, cause the computer system to perform inference-time operations, the inference-time operations comprising:
obtaining input data and one or more seed values associated with the input data;
processing the input data and the one or more seed values with the machine-learned model to generate one or more inferences from the input data, wherein the one or more seed values deterministically control which of the plurality of different processing operations are applied by the machine-learned model when processing the input data; and
providing the one or more inferences as an output.

2. The computer system of claim 1, wherein the input data comprises the one or more seed values or the one or more seed values are generated from the input data.

3. The computer system of claim 1, wherein:
the one or more seed values comprise a plurality of seed values;
processing the input data and the one or more seed values comprises respectively performing a plurality of inference runs of the machine-learned model with respect to the input data to generate a plurality of intermediate outputs, wherein the plurality of seed values are respectively used at the plurality of inference runs; and
providing the one or more inferences as the output comprises aggregating the plurality of intermediate outputs to generate an aggregated output.

4. The computer system of claim 1, wherein the one or more seed values have been selected using a hyperparameter optimization algorithm.

5. The computer system of claim 4, wherein the hyperparameter optimization algorithm comprises a simulated annealing algorithm.

6. The computer system of claim 1, wherein:
the input data comprises image data; and
the plurality of different processing operations comprise a plurality of different image augmentation operations.

7. The computer system of claim 1, wherein the input data comprises medical data and the one or more inferences comprise one or more diagnostic inferences.

8. The computer system of claim 1, wherein the input data comprises radiographic data.

9. The computer system of claim 1, wherein processing the input data and the one or more seed values with the machine-learned model to generate the one or more inferences from the input data comprises determining one or more values for one or more parameters of the machine-learned model based on the one or more seed values.

10. The computer system of claim 9, wherein determining the one or more values for the one or more parameters of the machine-learned model based on the one or more seed values comprises using a look-up-table or a pseudo-random number generator to determine the one or more values for the one or more parameters of the machine-learned model based on the one or more seed values.

11. A computer-implemented method for deterministic inference, the method performed by a computing system comprising one or more computing devices, the method comprising:
obtaining input data and one or more seed values associated with the input data;
accessing a machine-learned model, wherein the machine-learned model is configured to variably apply one or more of a plurality of different processing operations when processing an input;
processing the input data and the one or more seed values with the machine-learned model to generate one or more inferences from the input data, wherein the one or more seed values deterministically control which of the plurality of different processing operations are applied by the machine-learned model when processing the input data; and
providing the one or more inferences as an output.

12. The computer-implemented method of claim 11, wherein the input data comprises the one or more seed values.

13. The computer-implemented method of claim 11, wherein:
the one or more seed values comprise a plurality of seed values;
processing the input data and the one or more seed values comprises respectively performing a plurality of inference runs of the machine-learned model with respect to the input data to generate a plurality of intermediate outputs, wherein the plurality of seed values are respectively used at the plurality of inference runs; and
providing the one or more inferences as the output comprises aggregating the plurality of intermediate outputs to generate an aggregated output.

14. The computer-implemented method of claim 11, wherein the one or more seed values have been selected using a hyperparameter optimization algorithm.

15. The computer-implemented method of claim 14, wherein the hyperparameter optimization algorithm comprises a simulated annealing algorithm.

16. The computer-implemented method of claim 11, wherein:
the input data comprises image data; and
the plurality of different processing operations comprise a plurality of different image augmentation operations.

17. The computer-implemented method of claim 11, wherein the input data comprises medical data and the one or more inferences comprise one or more diagnostic inferences.

18. The computer-implemented method of claim 11, wherein processing the input data and the one or more seed values with the machine-learned model to generate the one or more inferences from the input data comprises determining one or more values for one or more parameters of the machine-learned model based on the one or more seed values.

19. The computer-implemented method of claim 18, wherein determining the one or more values for the one or more parameters of the machine-learned model based on the one or more seed values comprises using a look-up-table or a pseudo-random number generator to determine the one or more values for the one or more parameters of the machine-learned model based on the one or more seed values.

20. One or more non-transitory computer-readable media that collectively store:
a machine-learned model, wherein the machine-learned model is configured to variably apply one or more of a plurality of different processing operations when processing an input; and
wherein machine-learned model is configured to receive one or more seed values as input, wherein the one or more seed values deterministically control which of the plurality of different processing operations are applied by the machine-learned model when processing the input.
